(19) United States
(12) Patent Application Publication
(10) Pub. No.: US 2022/0276227 A1
(43) Pub. Date: Sep. 1, 2022

(54) Microfluidic Chip for Single Cell Pairing

(71) Applicant: Northeastern University, Boston, MA (US)

(72) Inventors: Yantao FAN, Malden, MA (US); Tania KONRY, Boston, MA (US)

(21) Appl. No.: 17/605,777

(22) PCT Filed: Apr. 30, 2020
(86) PCT No.: PCT/US2020/030886

§ 371 Date: Oct. 22, 2021

Related U.S. Application Data
(60) Provisional application No. 62/841,191, filed on Apr. 30, 2019.

(51) Int. Cl. G01N 33/50; C12Q 1/6851; B01L 3/00; C12M 3/06; C12M 1/00

(52) U.S. Cl. CPC G01N 33/5044; C12Q 1/6851; B01L 3/502761; C12M 23/16; C12M 47/04; B01L 2200/0652; B01L 2300/0864; B01L 2200/16; B01L 2300/0654; B01L 2300/0819; B01L 2400/0487

(57) ABSTRACT

A microfluidic system for high throughput characterization of interactions between pairs of cells is provided. A first cell is loaded into a capture chamber and transferred to a culture chamber, and then a second cell is captured and transferred to the same culture chamber, forming a pair of interacting or non-interacting but colocalized cells. The pair of cells can then be incubated, monitored by microscopy, and perfused with modulatory factors while interaction between the cells is investigated. The cells can be lysed, and whole cell lysates can be collected for genomic or proteomic analysis.

### CROSS REFERENCE TO RELATED APPLICATIONS

This application claims priority to U.S. Provisional Application No. 62/841,191, filed 30 Apr. 2019, the entirety of which is incorporated herein by reference.

### BACKGROUND

Heterotypic cell pairing and interaction has been characterized previously in different microfluidic platforms. Examples of immune-immune, cancer-immune, and immune-microbe co-encapsulation in droplets have been reported. Microwells and geometric structures such as pillars, traps, and weirs have been used to pair cells. However, recovery of precisely identified cell pairs for follow-up interrogation has required complicated micromanipulation, valves, and electrical or optical sorting techniques.

### SUMMARY

The present technology provides a microfluidic system for high throughput characterization of interactions between pairs of eukaryotic and/or prokaryotic cells. A first cell is loaded into a capture chamber and transferred to a culture chamber, and then a second cell is captured and transferred to the same culture chamber, forming a pair of interacting or non-interacting but co-localized cells. The pair of cells can then be incubated, monitored by microscopy, and perfused with modulatory factors while interaction between the cells is investigated. The cells can be lysed, and whole cell lysates can be collected for genomic or proteomic analysis.

The technology can provide methods for quickly pairing heterotypic cells. The pairing can be accomplished without artificially confining the cells and without forced interactions between the paired cells. Subsequent monitoring for natural interactions between a pair of living cells can be done. The interactions can be cell-initiated interactions. Pairs of cells can be individually monitored for interaction between the cells using various observation and measurement techniques, such as optical or fluorescence microscopy. Monitoring for an interaction can be accurately tuned to only detect cell-initiated interactions, and subsequent lysis of the pair of cells and detailed study is possible.

The present technology can be further summarized through the following features:

1. A microfluidic device for capture and pairing of two or more single cells, the device comprising:

a cell suspension inlet and a cell suspension outlet;

a first microfluidic channel fluidically connected at a first end to the inlet and at a second end to the outlet;

a working zone comprising a plurality of working units, each working unit comprising:
a portion of said first microfluidic channel;
a capture chamber fluidically connected to the first channel and a first pressure port;
a culture chamber fluidically connected to the first channel and a second pressure port;
   wherein the first channel provides a continuous fluid pathway from the cell suspension inlet through each working unit in sequence and then to the cell suspension outlet.
   2. The microfluidic device of feature 1, wherein each culture chamber is fluidically connected at opposite sides of the chamber to two microfluidic channels that in turn are each fluidically connected to the second pressure port.
   3. The microfluidic device of feature 2, wherein each of the two microfluidic channels comprises a constricted portion at its connection to the culture chamber, wherein a diameter of the constricted portion is smaller than a diameter of cells intended for capture in the capture chamber.
   4. The microfluidic device of any of the preceding features, wherein each capture chamber is fluidically connected, at a side opposite to the first channel, to a constricted channel that in turn is fluidically connected to the first pressure port, wherein a diameter of the constricted channel is smaller than a diameter of cells intended for capture in the capture chamber.
   5. The microfluidic device of any of the preceding features, wherein the width, depth, and height of the capture chambers and the culture chambers are each 2-fold to 20-fold time the average dimension of single cells intended for analysis in the device.
   6. The microfluidic device of feature 5, wherein the width, depth, and height of the capture chambers and the culture chambers are each in the range from 20 to 200 microns.
   7. The microfluidic device of any of the preceding features that permits light microscopic observation, imaging, spectrophotometric, and/or fluorescence analysis of cells in the capture and/or culture chambers of the device.
   8. The microfluidic device of any of the preceding features comprising at least 96 working units.
   9. The microfluidic device of any of the preceding features, wherein the second pressure port connected to each individual culture chamber has a separate fluidic connection to an individual port or collection chamber in the device or external to the device for the collection of cells or cell lysates from the culture chamber.
   10. The microfluidic device of any of the preceding features further comprising one or more paired single cells in a culture chamber of the device.
   11. A system for capture and pairing of single cells, the system comprising:

the microfluidic device of any of the preceding features;

a variable pressure fluid delivery device that is configured to provide negative or positive pressure individually to the first and second pressure ports of the microfluidic device;

optionally an imaging microscope system; and

optionally a processor, memory, and display for collection, analysis, and viewing of images or data from the microscope.

12. The system of feature 11, further comprising:

a separate fluid delivery device capable of flowing a cell suspension through the first microfluidic channel of the microfluidic device independently of the variable pressure fluid delivery device.

13. The system of feature 11 or 12, further comprising:

a controller capable of controlling fluid flow rate through the first microfluidic channel and/or capable of controlling pressure supplied by the variable pressure fluid delivery device.

14. A method for monitoring interaction between a pair of single living cells, the method comprising

(a) providing the system of any of features 11-13, wherein the system comprises an imaging microscope system;

(b) loading a first cell suspension through the cell inlet port of the microfluidic device and into the first microfluidic channel of the device

(c) capturing one single cell in each of one or more capture chambers of the device by applying negative pressure to the first pressure port;

(d) transferring the single cells from the one or more capture chambers to the adjacent culture chambers by applying negative pressure to the second pressure port;

(e) repeating steps (b) through (d) using a second cell suspension, resulting in formation of pairs of single first cells and single second cells in one or more culture chambers of the device;

(f) monitoring the cell pairs for interaction between the first and second cells in of each pair in the one or more culture chambers.

15. The method of feature 14, wherein steps (c) and/or (d) comprise stopping flow of cell suspension in the first microfluidic channel.
16. The method of feature 14 or 15, further comprising, between steps (d) and (e):

(d1) washing the first microfluidic channel to remove the first cell suspension.

17. The method of feature 16, wherein step (d1) comprises maintaining negative pressure at the first and/or second pressure ports during washing.
18. The method of any of features 14-17, further comprising:

(g) lysing one or both of the pair of cells by flowing a cell lysis solution through the first microfluidic channel and collecting the lysate through the second pressure port.

19. The method of any of features 14-18, wherein step (f) further comprises analysis of a cellular function or cellular composition of one or both of a pair of cells using the microscope system.
20. The method of feature 19, wherein the analysis comprises a determination of living or dead state; change in cell size, morphology, or motion; membrane potential; intracellular calcium concentration; presence, absence, or expression of one or more biomarkers; cell secretion; cytokine production or release, or response to a cytokine; expression level of a cell protein or gene; and/or increase, decrease, or stability of cell-cell contacts.
21. The method of any of features 14-20, wherein the first or second cell suspension comprises cells selected from the group consisting of cancer cells, natural killer cells, cytotoxic T cells, B lymphocytes, naive T cells, stem cells, bacterial cells, fungal cells, viral infected cells, single-celled microorganisms, and plant cells.
22. The method of any of features 14-21, wherein the first cell suspension comprises cancer cells and the second cell suspension comprises NK cells.
23. The method of feature 18, wherein the method further comprises genomic or proteomic analysis of the collected cell lysate.
24. The method of any of features 14-23, wherein one or both cells of a pair of cells is labelled with a unique label to allow individual monitoring of the cells.
25. The method of any of features 14-24, wherein the cells are monitored in step (f) for about 4 to 24 hours.

### BRIEF DESCRIPTION OF THE DRAWINGS

FIG. 1A shows a microfluidic device for the pairing of single cells. The working zone 505 is demarcated by a dashed line and is shown enlarged in FIG. 1B. Arrows 500 and 510 illustrate the transposition to FIG. 1B. The working zone shown in FIG. 1B has 96 working units, a portion of which are shown enlarged in FIG. 1C. Arrows 65 and 66 illustrate the transposition to FIG. 1C. FIG. 1D shows a diagram of a single working unit with the main channel or first channel 30 at center. FIG. 1E shows a light microscope image of a working unit from an actual device with the main channel or first channel 30 at center, the capture chamber 80 to the left of the channel, and culture chamber 85 to the right of the channel. FIG. 1F illustrates tuning dimensions of a working unit.

FIG. 2A shows a microscope image of a working unit of a microfluidic device as described in FIGS. 1A-1D prior to cell capture. FIG. 2B shows the same working unit as in FIG. 2A after capture of a first cell which is visible in the capture chamber. The arrow denotes fluid flow and the path of the captured cell. FIG. 2C shows the same working unit after transfer of the captured cell from the capture chamber to the culture chamber; the arrow denotes fluid flow. FIG. 2D shows the result of capture and transfer of a second cell, of a different type; in the same working unit. Arrows 68 and 69 show transposition of the culture chamber area to FIG. 2E, where it is enlarged. FIG. 2E shows an example of a non-interacting cell pair trapped within the culture chamber of a working unit of the device. FIG. 2F shows an example of an interacting cell pair in a similar working unit.

FIG. 3A shows cells in the culture chamber of a working unit before lysis. FIG. 3B shows addition of a lysis buffer in the main channel of the same working unit before lysis of the cells. FIG. 3C shows extraction of lysate material from the lysed cells under negative pressure induced flow. FIG. 3D shows the same working unit after lysate removal.

FIG. 4 shows results of a determination of pairing efficiency in several working units of a single 96-trap device. The left hand images show six different working units containing single cells in the culture chambers before pairing, and the right hand images show the same working units as on the left after pairing. Two-thirds of the traps are occupied by pairs of two different cell types. Three of the pairs are in contact while one is co-localized but not in contact.

### DETAILED DESCRIPTION

The present technology provides a negative pressure-driven cell entrapment and pairing microfluidic device with the capability of monitoring and modulating heterotypic pairs of eukaryotic cells, as well as recovering lysates from the individual cells for genomic or proteomic analysis. The device permits sequential capture and pairing of two different cell types with 60-80% efficiency. It also allows for stable, long term, dynamic monitoring of live cell-cell interactions. The culture chambers of the device are sufficiently large so they do not unduly confine cells or force interactions between cells. The device also does not immobilize cells with peptides or antibodies which can inadvertently trigger cell activation. The technology enables “bottom-up” analytical protocols that allow correlation of functional signatures related to cell interactions and single-cell dynamics with genetic signatures, providing a comprehensive overview of cellular machinery.

An example of cell-cell interaction that can be characterized using the present microfluidic device is that between a natural killer cell (NK cell) and a cancer cell. NK cells physically interact with and determine whether or not to lyse target cancer cells. Due to the innate heterogeneity of both cancer and NK cells, the functional outcome of individual NK cell-cancer cell interaction varies widely. Thus, single cell analysis serves as a promising strategy to dissect the variability in effector-target (E-T) interactions and subsequently correlate them to cell-specific bioelectric fingerprints, for example. Such analysis can facilitate a better understanding of endogenous voltage dependency on immunogenic interactions in the TME (tumor microenvironment). The analysis also can be used to promote reprogramming of NK cell immunotherapeutic efficacy. Toward this end, a scalable hydrodynamic microfluidic device is described herein having the following capabilities: (a) efficient formation of trapped cell pairs, such as NK cell-cancer cell pairs, in a 1:1 ratio and in a fast, controllable manner; (b) time-lapse monitoring of dynamic cellular interactions, including voltage gradients and cytolysis; and (c) on-chip lysis and retrieval of intracellular components for end-stage genomic/proteomic profiling.

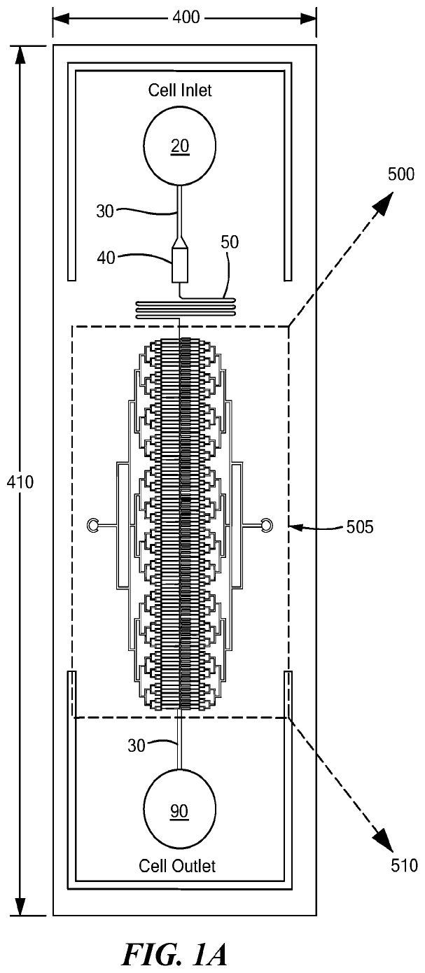

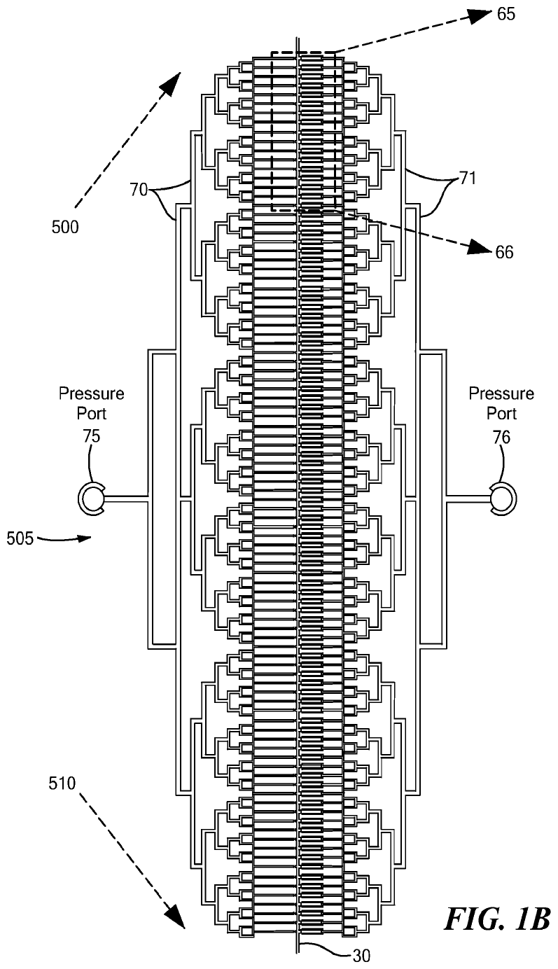

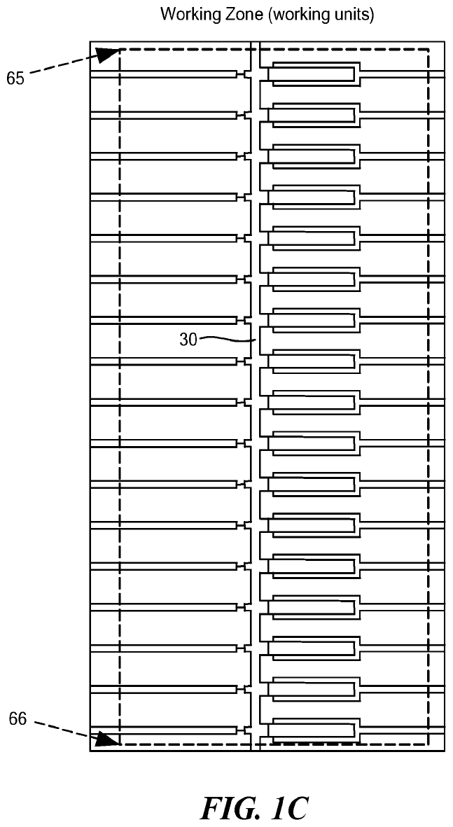

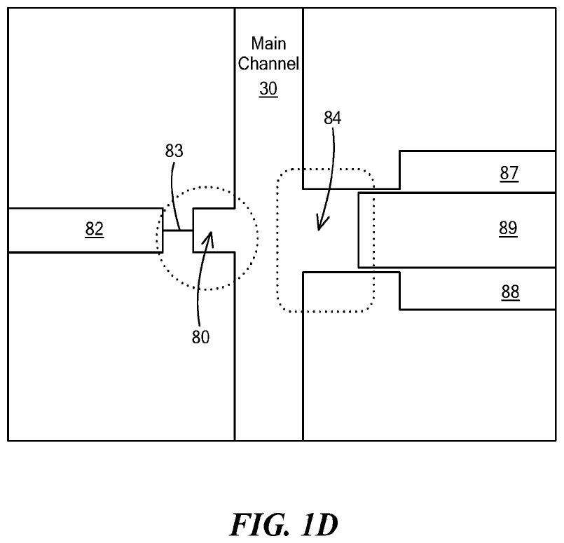

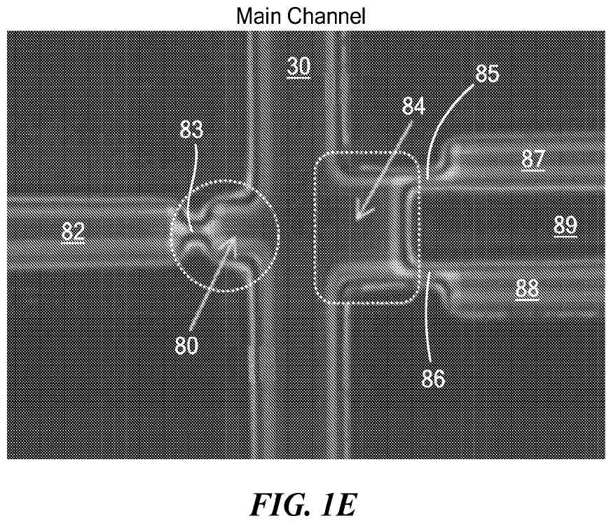

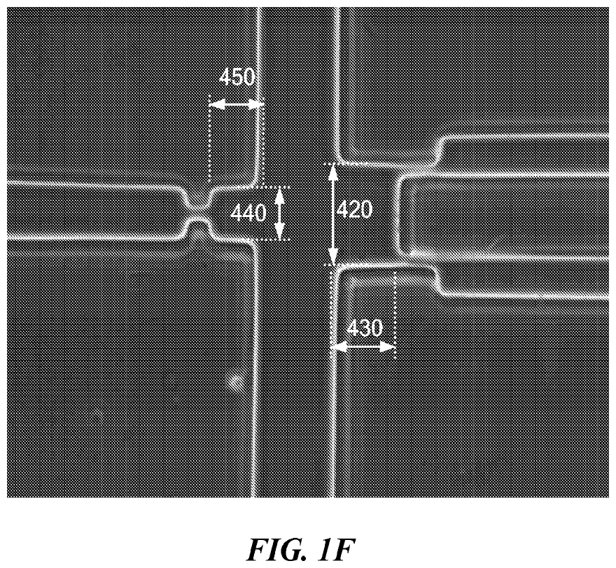

FIG. 1A shows a diagram of a scalable microfluidic device, or “chip”, with an overall (scalable) illustrated length 410 and a width 400. The device includes cell inlet 20 and cell outlet 90, each of which can provide fluidic connection through tubing or other structures to reservoirs for cell suspensions flowing into or out of the device. The device has a single main channel (or “first channel”) 30, which is a microfluidic channel allowing a cell suspension to flow through the working units of the device. Working zone 505, enclosed by a dashed line in FIG. 1A, has 96 working units in this design, although the number of working units can be scaled larger or smaller as desired. The chip is scalable, and length 410 and width 400 can be, for example, any desired value in the millimeter or centimeter ranges. The chip can have an overall oval shape, as shown in FIG. 1A, or it can be circular or have another desired geometry. Cell inlet reservoir 20 is in fluid connection with first channel 30. Optional filter 40 and cell spacing extension 50 are shown. The optional cell spacing extension 50 can be utilized to identify the amount of spacing or regularity of spacing between cells as they flow along the extension. First channel 30 makes fluid connection between cell inlet 20 and the working zone 505, which contains, for example, 96 working unit. Downstream of the working zone, channel 30 makes fluid connection and outlet 90. The device can be made of any suitable material, such as, for example, polydimethylsiloxane (PDMS), polymethyl methacrylate (PMMA), polystyrene, polycarbonate, and/or glass.

The working zone shown in FIG. 1B illustrates pressure ports 75 and 76 for the selective application of negative pressure, i.e., vacuum, or positive pressure in the form of a pressure-regulated fluid, such as an isotonic saline solution, at either the cell capture (75) or cell culture (76) ends of the working units. The capture gas outlet 75 is in fluid communication with capture connecting microchannels 70, which lead to the capture connecting channel 82 shown in FIG. 1D. By utilizing multiple capture outlets (not shown in FIG. 1B), each capture connecting channel 82, shown in FIG. 1D, can have an associated capture outlet, without capture connecting microchannels 70. Similarly, culture gas outlet 76 is in fluid communication with culture connecting microchannels 71, which lead to culture connecting channels 87 and 88 shown in FIG. 1D. Multiple culture outlets (not shown in FIG. 1B) can be utilized, so that each pair of culture connecting channels 87 and 88 has an associated culture outlet without culture microchannels 71.

Pressure, either negative or positive, can be applied independently at pressure ports 75 and 76, through any suitable regulated pump or vacuum source capable of supplying a fluid or gas at a desired pressure and very low or essentially no flow rate, sufficient to manipulate single cells in the microfluidic device.

In FIG. 10 about seventeen of the 96 working units from FIG. 1B are enlarged to show details of the working units and first channel 30. The capture outlet 75 or the culture outlet 76 can be utilized as inlets as needed for pressure equalization. First channel 30 is central to several working units. Each working unit has two functionally distinct sites: capture chamber 80 and culture chamber 84 (see also FIGS. 1D and 1E). Capture connecting channel 82 is in fluid communication with capture constriction 83, which fluidically connects to first channel 30 through capture chamber 80. Culture chamber 84 is fluidically coupled through first culture constriction 85 and second culture constriction 86 with culture connecting channels 87 and 88, respectively. The first and second culture constrictions are structurally and functionally equivalent, but each can access a different cell in non-contact pairings. In FIGS. 1D and 1E, separation structure 89 separates culture connecting channels 87 and 88.

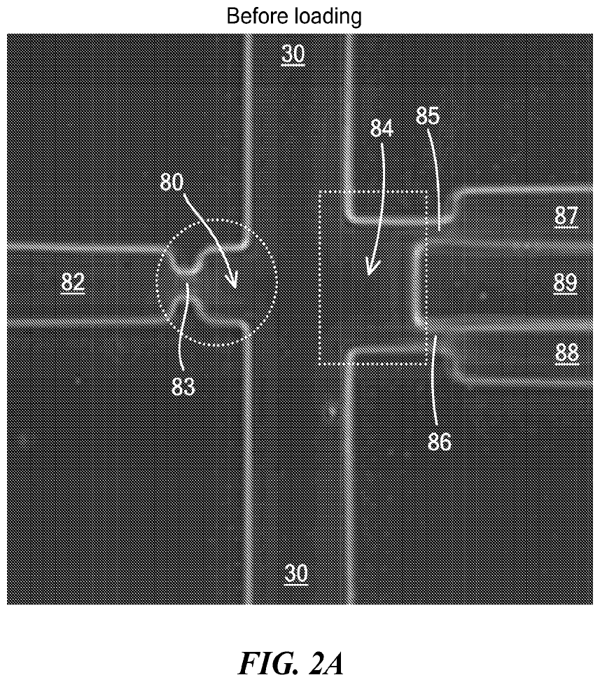

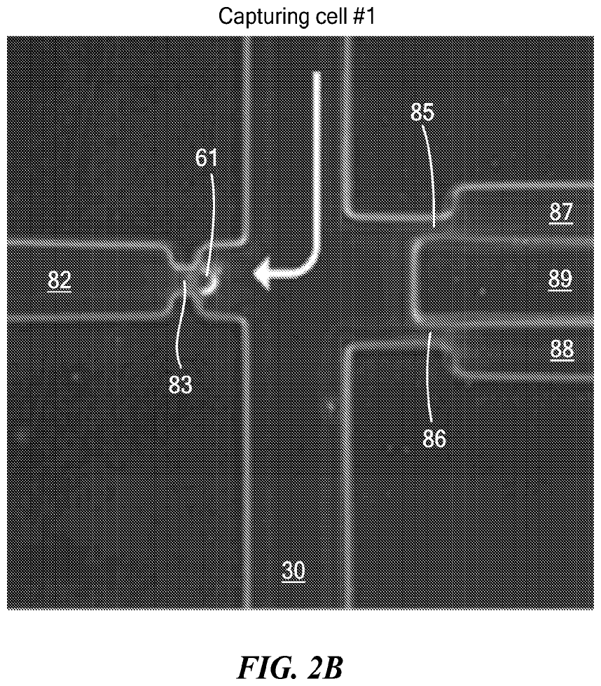

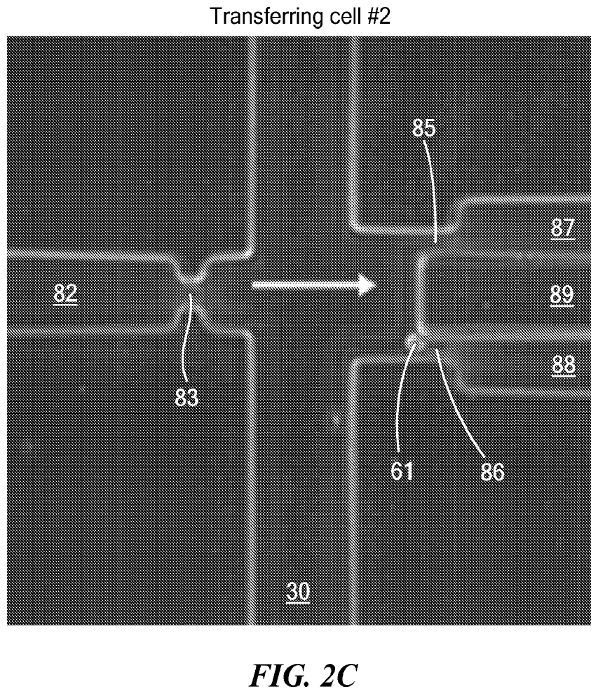

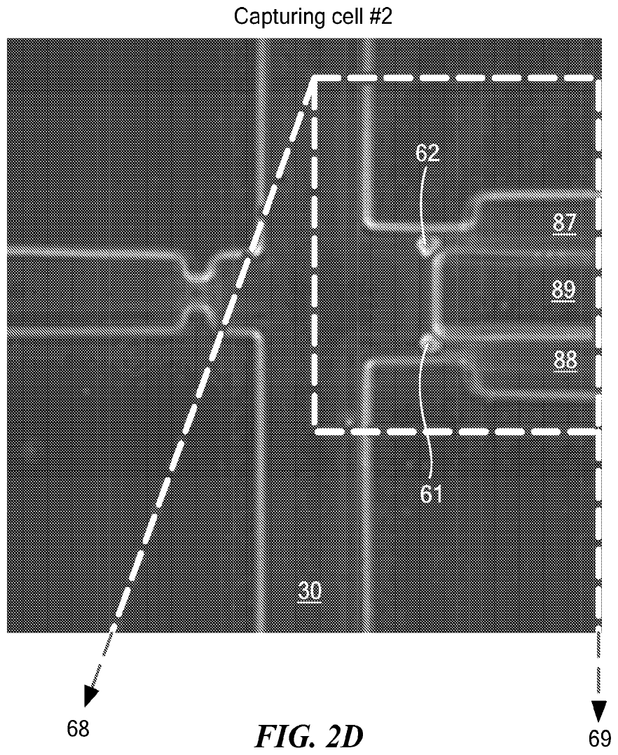

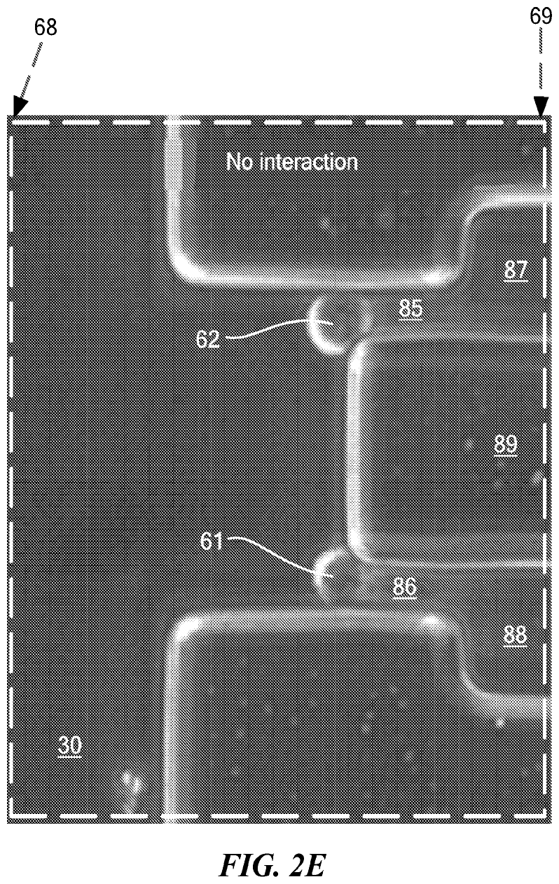

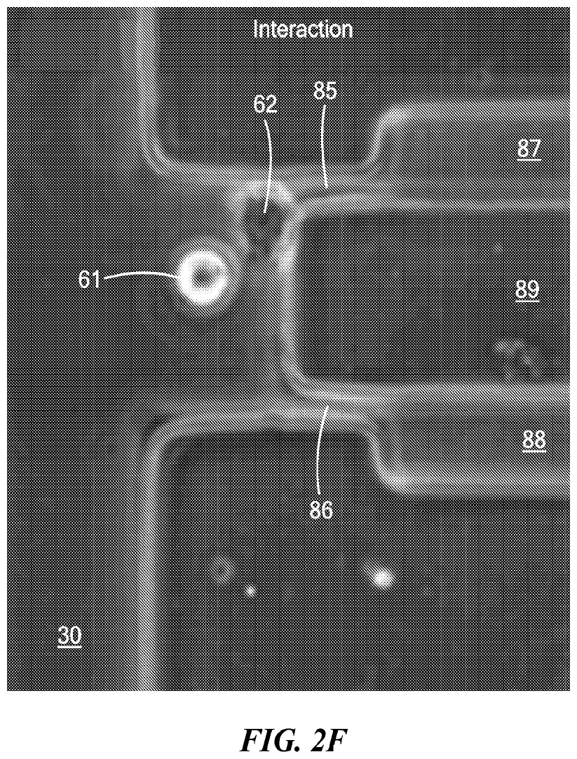

In FIG. 1F, the width of the culture chamber is shown as 420, and the depth of the culture chamber is shown as 430. The width of the capture chamber is shown as 440, and the depth of the capture chamber is shown as 450. An additional dimension which is not shown is the height of each chamber, which may be similar to any of the dimensions 420, 430, 440, 450, or a different height, preferably in the range of sever multiples (e.g., 2×, 3×, 4×, 5×, 6×, 7×, 8×, 9×, 10×, 15×, or 20×) of the anticipated diameter of the cells under study. The culture chamber width 420 can be sufficiently wide to that one or two cells deposited at culture constrictions 85 and 86 (see FIG. 1E) are not constrained, deformed, or mechanically forced into contact. For example, the culture chamber width can be about 2-20 times the diameter of a cell to be studied, such as about 2, 3, 4, 5, 6, 7, 8, 9, 10, 15, or 20 times the diameter. A pair of cells in the culture chamber (see 61 and 62 in FIG. 2D) can be brought into proximity and then monitored for cell-initiated interactions (see FIG. 2F). The culture chamber depth 430 can be sufficiently large so that cells in the culture chamber are not adversely affected or activated by flow through central channel 30 yet sufficiently small so that cells can be easily transferred from capture chamber to culture chamber and are likely to interact. The capture chamber width 440 can be can be about 2-20 times the diameter of a cell to be studied, such as about 2, 3, 4, 5, 6, 7, 8, 9, 10, 15, or 20 times the diameter. The capture chamber depth 450 can be sufficiently large so that cells in the capture chamber are not dislodged by flow through central channel 30 yet sufficiently small so that cells can be easily transferred from capture chamber to culture chamber by fluid flow between the chambers. By tuning these dimensions, as shown in FIG. 1F, paired cells can be brought into close proximity without forcing the cells into direct physical contact. With appropriate dimensions, a cell that initiates interaction with another cell can cease that interaction and move away in some cases.

Heterotypic cell loading, entrapment, and docking can be conducted sequentially, thus ensuring deterministic pairing. First, a suspension containing cell type #1 (e.g., cancer cells) is injected into the main channel through the inlet (FIGS. 2A-2B) and captured in the traps by application of negative pressure (FIG. 2B). Fluid flow conditions and trap geometry are optimized to ensure single cell trapping in each array and to prevent overloading. Once cells of a first type have been loaded into the array, the remaining cells in the main channel can be flushed out through the first channel with a washing buffer (e.g., 1×PBS). The trapped cells are then transferred to the culture chamber by selective implementation of negative pressure at the culture chamber side of the device. Preferably, negative pressure in the capture chambers is switched off, or made positive, to release the trapped cells at the same time that negative pressure is switched on in the culture chambers to draw in the released cells (FIG. 2C). Negative pressure or positive pressure can be used depending on configuration and desired movement of cells. After the first cell type is secured in the culture chamber (FIG. 2C), which has been made with a culture depth deep enough to hold cells even in the absence of negative pressure, a suspension of cell type #2 (e.g., NK cells) are introduced and trapped in the capture chambers and subsequently transferred to the culture chambers in the same way as for the first cells (FIG. 2D). At the end of the loading process, single first type cells are paired with single second type of cells, for example, NK cells, in the 96-well array. The cells can be either paired in contact or not in contact but within the same culture chamber (i.e., co-localized). The device is scalable by design, as the throughput can be increased easily by adding more working units. The culture medium inlet and outlet can supply continuous or intermittent flow of culture medium, or for the delivery of biological or chemical agents to activate or modulate any desired cellular process or function or to stimulate or inhibit cell-cell interactions. This can provide for long-term maintenance, monitoring, and stimulation or modulation of the cultured cell pairs (FIGS. 2E and 2F). Monitoring can be for hours or days, so long as the entrapped cell pairs are cultured. The device can be transparent at least on one side such that optical microscopy, including fluorescence microscopy and other forms of microscopy, can be performed to characterize cell function during monitoring. FIG. 2E shows paired cells with no cell-cell contact interactions after pairing. FIG. 2F shows a cell pair interacting though close contact.

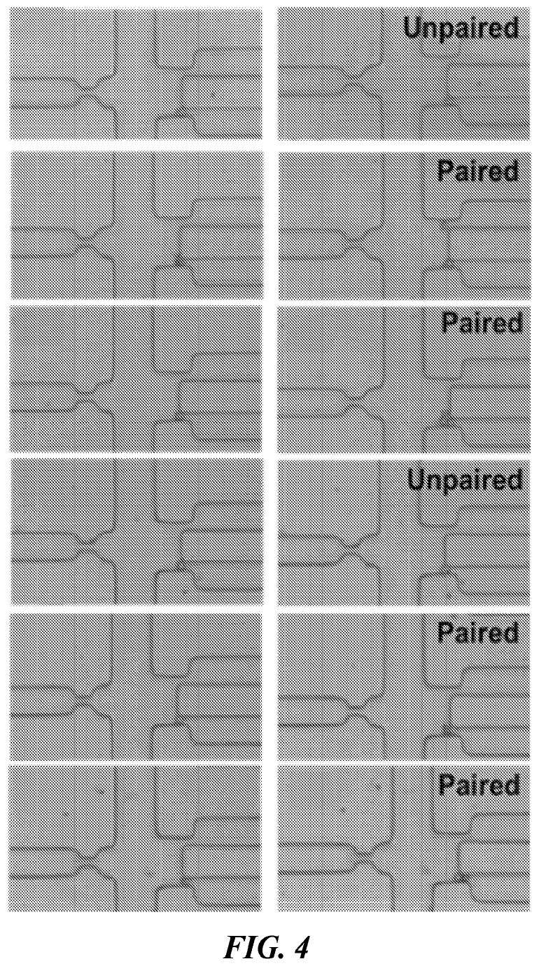

The efficiency of cell pairing is closely related to the design of the capturing and culturing chamber. To maximize pairing efficiency, the dimensions of these structures are optimized by testing various widths, lengths, and depths of each trap. For example, considering the diameter of a single cell to be about 10 μm, the capture chamber width and depth can be set to 30 μm×30 μm respectively. The culture chamber can be designed, for example, to be about 60 μm in width and about 30 μm in depth. Capture chamber depth is illustrated as dimension 450 in FIG. 1F. Capture chamber width is illustrated as dimension 440 in FIG. 1F. Culture chamber depth is illustrated as dimension 430 in FIG. 1F. Culture chamber width is illustrated as dimension 420 in FIG. 1F. The experimentally determined dimensions, for example, resulted in a capture and pairing efficiency of 50-60% (FIG. 4). Pairing efficiency can be increased to about 80% by reducing channel width, trap dimension, and altering cell seeding density. The flow-driven pressure ranges that enables an efficient single cell capture without affecting the cell's viability was also optimized. The loading time for each cell type was found to vary from 8-10 min. This time can be reduced, for example, by implementing a high-density trap array. The loading time can be about 1 min., about 2 minutes, about 3 minutes, about 4 minutes, about 5 minutes, about 6 minutes, about 7 minutes, about 8 minutes, about 9 minutes, or about 10 minutes or longer, depending on the experiment.

The number of working units on a single device can be scaled to as desired (e.g., 12, 24, 96, 384, or 1536 working units per device) to improve throughput or align it with the quantity of testing or availability of cells or reagents to be tested. It is believed that all recovery outlets can be seamlessly integrated with the capture/culture chambers to allow recovery of multiple cell extracts from the platform for correlated genotypic/phenotypic analysis.

Cell function parameters that can be analyzed include any cell function parameters typically determined for single cells, including but not limited to: living or dead state; changes in cell size, morphology, or motion; membrane potential; intracellular calcium concentration; presence, absence, or expression of one or more biomarkers; cell secretion; cytokine production or release, or response to cytokines; expression of cell surface proteins; increase, decrease, or stability of cell-cell contacts; and the like. A large number of knows methods can be applied to determine or monitor these and other features of single cells of a pair.

### Dynamic Monitoring and On-Chip Lysis of Assayed Cells

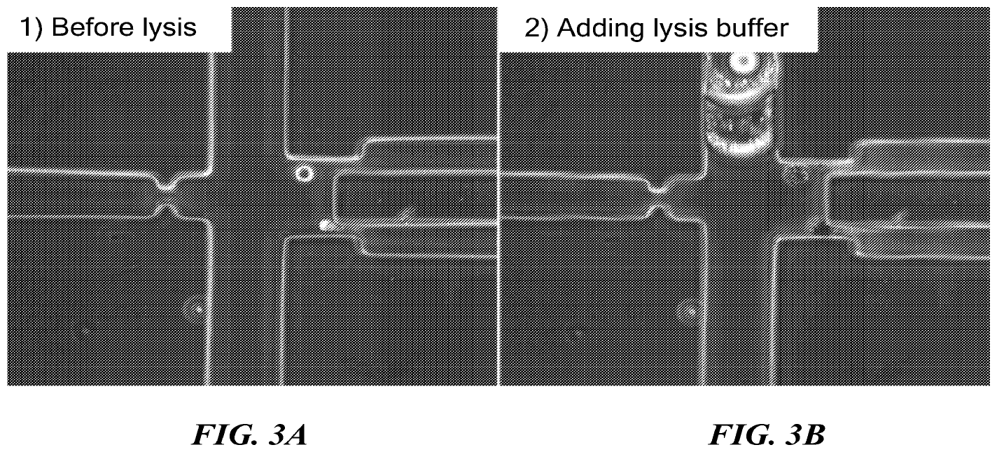

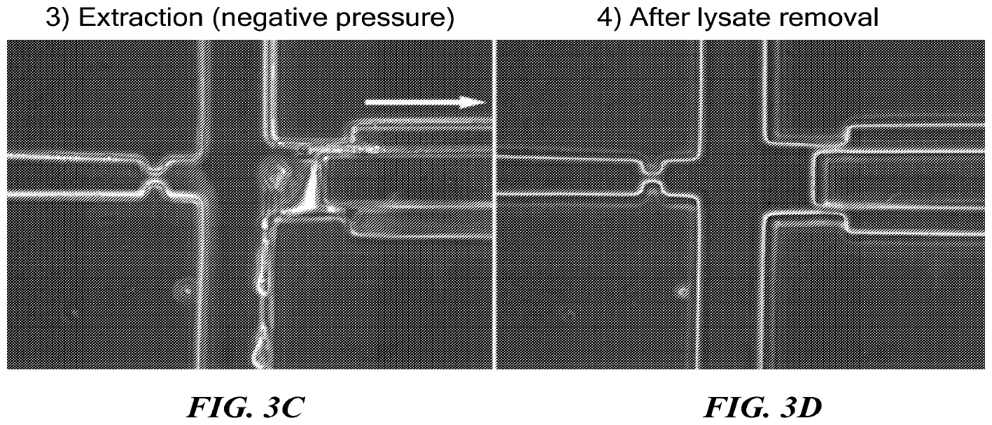

The present technology can monitor the docked cells over varying experimental duration (e.g., 4-24 hours or longer) and then chemically lyse the monitored cells on-chip for downstream analysis. The entrapped cells can be cultured in the reservoirs over prolonged periods as fresh culture media is provided continually through the inlet of the device. Likewise, the cells can also be stimulated by introducing activating reagents in the media to assess their response. The identity of the heterotypic cells can be preserved by fluorescent labeling with two types of cell tracker dyes. The cells can be dynamically monitored over time to determine various stages of E-T interaction-conjugation, elongation, dissociation, and/or cytolysis. At the end of the interactive period, the cells are chemically lysed using a lysis buffer in each culture chamber to preserve the identity of individual cell pairs and correlate genetic markers with functional signatures. To this end, single culture constrictions (of 2-4 μm width) corresponding to each culture chamber can been introduced. Single culture constrictions 85 and 86 are shown, for example, in FIG. 2F. The width of the constriction can be varied from 1-5 μm to provide a balance between stable entrapment and lysate recovery. Optionally, the width of a capture or culture constriction can be 1 μm, 2 μm, 3 μm, 4 μm, 5 μm, 6 μm, 7 μm, 8 μm, 9 μm, or 10 μm, or about 15 μm, and specified ranges can be designated by combining, for example, 2 μm to about 8 μm. The device is scalable depending upon the study. The cell size or diameter is compared to a proposed constriction. To isolate total cellular content, the traps and channels can be first washed three times with PBS. If the cancer cell, for example, has been killed by the NK cell, for example, during E-T interaction, the dead cancer cell is washed away, leaving the single NK cell in the culture chamber. Lysis buffer is loaded into the main channel through the inlet (see FIG. 3B). After a short 5 min interval to permit lysis, negative pressure is applied to the culture chamber to collect the whole single-cell extract (see FIG. 3C).

The device described herein may be used to assess cancer-immune cell interaction and effector functions under precisely controlled conditions. As the first step toward achieving this objective, the viability of entrapped cells is characterized to rule out the possibility, however unlikely, of damage due to loading and transfer in the various chambers. Each cell type, for example, cancer and NK cells, can be individually assessed in the microfluidic platform. The cells can be labeled with calcein AM (live cell indicator), while ethidium homodimer (death indicator) is added to the solution (Life Technology) in the device in a humidified stage-top incubator at about 37° C. with 5% CO2. Cells can be also seeded in 96-well plates as a control and cell viability monitored hourly. Data obtained can indicate minimal cell death of each cell type (cancer and NK cell) over a period of 24 hours.

Paired cells (FIG. 4B) can be quantified to compare, for example, spontaneous target cell death with NK-mediated target cytolysis. At one-to-one level, the interaction of NK cells with target cells may be contact-dependent, that is, through formation of activating or inhibitory immune synapse, or contact-independent, for example, through cytokine and/or exosome secretion.

Both types of interaction can be characterized using, for example, NK-92 cell line, which is currently under investigation as a cellular anti-tumor therapy. NK92 (CD56+CD16+) cells have been characterized in preclinical models extensively and shown promising results in four phase I trials worldwide for different cancers. NK-92 cells can be paired with K562 cells, a typical NK cell target, to determine the quantitative functional features of their interaction. Contact-dependent interaction profiles can be interrogated based on (a) contact duration, (b) frequency of contact, (c) rate of association/dissociation, (d) change in motility, and (e) timings of NK-mediated cancer cell lysis. Significant heterogeneity can be detected in all these quantitative parameters at the single cell level, which may be used to establish distinct phenotypic subsets with graded levels of activity. For instance, “responsive” cancer cells may be killed in the microfluidic platform rapidly (<30 min) while “nonresponsive” cancer cells may not be killed by NK cells or killed after prolonged entrapment (>4 hrs; temporal thresholds can be defined experimentally).

Correlation of Functional Features with Genetic Profile Via Single Cell qPCR

The recovered lysates (FIG. 3C, FIG. 3D) can be subjected to single cell qPCR analysis to assess changes in known NK cell markers so as to validate the performance of the cell-pairing chip. Functional NK cell markers (e.g., CD107a, IFN-γ, TNF, granzyme B, and perforin) can be tested in this panel. NK cells that have interacted with and killed target cancer cells are expected to upregulate several molecules including granzyme B and perforin, while NK cells that have not killed target cells or have not interacted with target cells will not increase expression of these markers. The lysates can be used to obtain total RNA, for example, by using the RNeasy Microkit without DNase treatment (Qiagen). Purified RNA can be used for reverse transcription and quantitative real-time PCR (qPCR) performed, for example, on the LightCycler 480 (Roche) using commercially available master mixes. The qPCR conditions have been optimized and, for example, they are: 3 min at 95° C. followed by 45 cycles of amplification (95° C. for 20 s, 58° C. for 20 s, and 72° C. for 20 s). All samples can be analyzed by melting curve analysis (60-95° C. at 0.1° C. continuous increments). Products of the PCR can be confirmed by agarose gel electrophoresis. Cycle of quantification (Cq) values can be obtained by the maximum second derivative method. The extracted RNA can be used for an unbiased transcriptome-wide analysis and classification of single cells, such as that performed with a protocol based on SMART-Seq2 cDNA and Illumina Nextera XT LC.

The present technology allows scale-up via parallelization and integration. The methodology and device are mild enough to co-encapsulate different types of mammalian cells along with bioassay reagents with high viability. The device and method also allow simultaneous and dynamic interrogation of thousands of paired cells in a short period of time and in one device or a few devices. The dynamic monitoring of cellular activity, e.g., kill target cells in the proposed droplet microarray, can define the time points for classification of NK cells into ‘hyperactive’ and ‘basal’ cells depending upon rapidity of response. The device and methods can be used for personalized medicine and to identify and select appropriate cancer therapies.

### Recovery of Lysates of a Specific Cell Pair

The device design and on-chip lysis protocol were tested for single working units. Following repeated washes, 20 μL lysis buffer (Thermo Fisher) was loaded into the unit (FIG. 3B) and maintained at room temperature for 5 minutes (FIG. 3C). The lysate was moved to a collection outlet (e.g., culture outlet) by negative pressure (FIGS. 3C and 3D) and frozen at −80° C. for analysis at a later time. Up to 96 NK cell extracts were recovered from every loaded unit after 4-24 hrs of entrapment, proving the reproducibility of this approach.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expressions “consisting essentially of” or “consisting of”.

What is claimed is:

1. A microfluidic device for capture and pairing of two or more single cells, the device comprising:
a cell suspension inlet and a cell suspension outlet;
a first microfluidic channel fluidically connected at a first end to the inlet and at a second end to the outlet;
a working zone comprising a plurality of working units, each working unit comprising:
a portion of said first microfluidic channel;
a capture chamber fluidically connected to the first channel and a first pressure port;
a culture chamber fluidically connected to the first channel and a second pressure port;
wherein the first channel provides a continuous fluid pathway from the cell suspension inlet through each working unit in sequence and then to the cell suspension outlet.

2. The microfluidic device of claim 1, wherein each culture chamber is fluidically connected at opposite sides of the chamber to two microfluidic channels that in turn are each fluidically connected to the second pressure port.

3. The microfluidic device of claim 2, wherein each of the two microfluidic channels comprises a constricted portion at its connection to the culture chamber, wherein a diameter of the constricted portion is smaller than a diameter of cells intended for capture in the capture chamber.

4. The microfluidic device of claim 1, wherein each capture chamber is fluidically connected, at a side opposite to the first channel, to a constricted channel that in turn is fluidically connected to the first pressure port, wherein a diameter of the constricted channel is smaller than a diameter of cells intended for capture in the capture chamber.

5. The microfluidic device of claim 1, wherein the width, depth, and height of the capture chambers and the culture chambers are each 2-fold to 20-fold time the average dimension of single cells intended for analysis in the device.

6. The microfluidic device of claim 5, wherein the width, depth, and height of the capture chambers and the culture chambers are each in the range from 20 to 200 microns.

7. The microfluidic device of claim 1 that permits light microscopic observation, imaging, spectrophotometric, and/or fluorescence analysis of cells in the capture and/or culture chambers of the device.

8. The microfluidic device of claim 1 comprising at least 96 working units.

9. The microfluidic device of claim 1, wherein the second pressure port connected to each individual culture chamber has a separate fluidic connection to an individual port or collection chamber in the device or external to the device for the collection of cells or cell lysates from the culture chamber.

10. The microfluidic device of claim 1 further comprising one or more paired single cells in a culture chamber of the device.

11. A system for capture and pairing of single cells, the system comprising:
the microfluidic device of claim 1;
a variable pressure fluid delivery device that is configured to provide negative or positive pressure individually to the first and second pressure ports of the microfluidic device;
optionally an imaging microscope system; and
optionally a processor, memory, and display for collection, analysis, and viewing of images or data from the microscope.

12. The system of claim 11, further comprising:
a separate fluid delivery device capable of flowing a cell suspension through the first microfluidic channel of the microfluidic device independently of the variable pressure fluid delivery device.

13. The system of claim 11, further comprising:
a controller capable of controlling fluid flow rate through the first microfluidic channel and/or capable of controlling pressure supplied by the variable pressure fluid delivery device.

14. A method for monitoring interaction between a pair of single living cells, the method comprising
(a) providing the system of claim 11, wherein the system comprises an imaging microscope system;
(b) loading a first cell suspension through the cell inlet port of the microfluidic device and into the first microfluidic channel of the device
(c) capturing one single cell in each of one or more capture chambers of the device by applying negative pressure to the first pressure port;
(d) transferring the single cells from the one or more capture chambers to the adjacent culture chambers by applying negative pressure to the second pressure port;
(e) repeating steps (b) through (d) using a second cell suspension, resulting in formation of pairs of single first cells and single second cells in one or more culture chambers of the device;
(f) monitoring the cell pairs for interaction between the first and second cells in of each pair in the one or more culture chambers.

15. The method of claim 14, wherein steps (c) and/or (d) comprise stopping flow of cell suspension in the first microfluidic channel.

16. The method of claim 14, further comprising, between steps (d) and (e):
(d1) washing the first microfluidic channel to remove the first cell suspension.

17. The method of claim 16, wherein step (d1) comprises maintaining negative pressure at the first and/or second pressure ports during washing.

18. The method of claim 14, further comprising:
(g) lysing one or both of the pair of cells by flowing a cell lysis solution through the first microfluidic channel and collecting the lysate through the second pressure port.

19. The method of claim 14, wherein step (f) further comprises analysis of a cellular function or cellular composition of one or both of a pair of cells using the microscope system.

20. The method of claim 19, wherein the analysis comprises a determination of living or dead state; change in cell size, morphology, or motion; membrane potential; intracellular calcium concentration; presence, absence, or expression of one or more biomarkers; cell secretion; cytokine production or release, or response to a cytokine; expression level of a cell protein or gene; and/or increase, decrease, or stability of cell-cell contacts.

21. The method of claim 14, wherein the first or second cell suspension comprises cells selected from the group consisting of cancer cells, natural killer cells, cytotoxic T cells, B lymphocytes, naive T cells, stem cells, bacterial cells, fungal cells, viral infected cells, single-celled microorganisms, and plant cells.

22. The method of claim 14, wherein the first cell suspension comprises cancer cells and the second cell suspension comprises NK cells.

23. The method of claim 18, wherein the method further comprises genomic or proteomic analysis of the collected cell lysate.

24. The method of claim 14, wherein one or both cells of a pair of cells is labelled with a unique label to allow individual monitoring of the cells.

25. The method of claim 14, wherein the cells are monitored in step (f) for about 4 to 24 hours.
